(19) United States
(12) Patent Application Publication
(10) Pub. No.: US 2005/0214371 A1
(43) Pub. Date: Sep. 29, 2005

(54) Stable pharmaceutical composition comprising an acid labile drug

(76) Inventors: Simona Di Capua, Kfar Saba (IL); Nava Shterman, Petach Tikva (IL); Limor Ari-Pardo, Israel (IL); Esther Itah, Netanya (IL)

Correspondence Address: KENYON & KENYON, ONE BROADWAY, NEW YORK, NY, 10004, US

(21) Appl. No.: 11/068,881

(22) Filed: Mar. 2, 2005

Related U.S. Application Data
(60) Provisional application No. 60/549,653, filed on Mar. 3, 2004.

(51) Int. Cl. A61K 31/4439; A61K 9/24

(52) U.S. Cl. 424/472; 514/338

(57) ABSTRACT

The present invention provides a stable pharmaceutical composition of an acid labile drug such as a pharmaceutically active substituted benzimidazole compound, comprising: a) an inner core coated with the acid labile drug; b) a first intermediate coating devoid of an alkaline stabilizing agent and the benzimidazole compound; c) a second intermediate coating comprising an alkaline stabilizing agent; and, d) an outer enteric layer. The present invention also provides a method of preparing the same.

### CROSS REFERENCE TO RELATED APPLICATION

This application claims the benefit of U.S. Provisional Application No. 60/549,653 filed on Mar. 3, 2004, the disclosure of which is incorporated by reference in its entirety.

### FIELD OF THE INVENTION

The present invention relates to stable pharmaceutical compositions. More particularly, this invention provides a stable pharmaceutical composition comprising solid carriers for an acid labile drug such as a pharmaceutically active substituted benzimidazole compound and methods of preparing the same.

### BACKGROUND OF THE INVENTION

Substituted 2-(2-pyridylmethyl) sulfinyl-1H-benzimidazoles are known gastric proton pump inhibitors. Lansoprazole is a substituted benzimidazole compound effective in inhibiting gastric acid secretion. This drug is used for the treatment of gastric and duodenal ulcers, severe erosive esophagitis, Zolinger-Ellison syndrome and H. pylori eradication. Other substituted 2-(2-pyridylmethyl) sulfinyl-1H-benzimidazole compounds, which are proton pump inhibitors effective in treating these diseases, include omeprazole, pantoprazole, rabeprazole, esomeprazole, hydroxyomeprazole, pariprazole, perprazole and tenatoprazole. Leminoprazole, which is a substituted 2-(phenylmethyl) sulfinyl-1H-benzimidazole compound, is also a proton pump inhibitor effective in treating these diseases.

Lansoprazole per se is disclosed in U.S. Pat. No. 4,628,098 assigned to Takeda Chemical Industries, Ltd. It is known chemically as (2-[[[3-methyl-4-(2,2,2-trifluoro-ethoxy)-2-pyridinyl]methyl]sulfinyl]-1H-benzimidazole) and has the following chemical formula A: 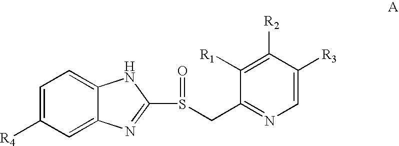
wherein R₁ is methyl, R₂ is trifluoro-ethoxy, and R₃ is hydrogen and R₄ is hydrogen. Omeprazole and pantoprazole share lansoprazole's ability to inhibit gastric acid secretion.

It is well known that substituted 2-(2-pyridylmethyl) sulfinyl-1H-benzimidazole compounds and leminoprazole have poor stability when exposed to acidic conditions. The stability decreases with decreasing pH. For example, the half-life of an aqueous lansoprazole composition in an acidic condition (pH of 5) is on the order of about 30 minutes, whereas in a neutral condition (pH of 7) the half-life is on the order of 18 hours. Furthermore, the stability of these substituted benzimidazole compounds is adversely affected by heat and moisture.

Substituted 2-(2-pyridylmethyl) sulfinyl-1H-benzimidazole compounds and leminoprazole are acid labile. These compounds are often designed in an enteric coated dosage form to avoid degradation of the active pharmaceutical ingredient (API) at the low pH found in the stomach. However, because enteric coatings are generally comprised of acidic compounds, direct covering of the substituted benzimidazole compounds with these types of coatings may cause degradation and decomposition of the API, causing the active pharmaceutical ingredient preparation to undergo discoloration and to lose its active ingredient content over time.

Attempts have been made to address the stability problem. One method involves incorporating an alkaline reacting agent into the substituted 2-(2-pyridylmethyl) sulfinyl-1H-benzimidazole preparations. For example, U.S. Pat. Nos. 4,628,098, 5,026,560, 6,296,875, 6,123,962, 6,017,560, 5,879,708, 6,639,478, 5,433,959, 5,093,132, 4,689,333 and 5,045,321 disclose a stable pharmaceutical composition comprising a substituted 2-(2-pyridylmethyl) sulfinyl-1H-benzimidazole compound and an inorganic alkaline salt. The inorganic alkaline salt in these preparations is in “even contact” with the substituted 2-(2-pyridylmethyl) sulfinyl-1H-benzimidazole compound.

U.S. Pat. No. 5,626,875 discloses a pharmaceutical formulation comprising a substituted 2-(2-pyridylmethyl) sulfinyl-1H-benzimidazole compound that is devoid of an alkaline stabilizer. Instead, a non-alkaline isolation layer is used to separate the core containing benzimidazole compounds from the acidic enteric coat.

“Stabilized Pharmaceutical Formulation of an Acid Labile Benzimidazole Compound and Its Preparation” (published in the October 2002 issue of The IP.com Journal) discloses a stable pharmaceutical formulation in the form of a multi-particulate delivery system. The system comprises: a) an inert core coated with a substituted 2-(2-pyridylmethyl) sulfinyl-1H-benzimidazole compound; b) an intermediate coating comprising an alkaline reacting agent; and, c) an outer enteric layer. There is no separation between the drug-containing layer and the intermediate coating that contains any alkaline stabilizing agent.

There is a continuing need for a stable pharmaceutical composition containing an acid-labile drug, such as a substituted 2-(2-pyridylmethyl) sulfinyl-1H-benzimidazole compound or a substituted 2-(phenylmethyl)sulfinyl-1H-benzimidazole compound, and a method of preparing therefor.

### SUMMARY OF THE INVENTION

The present invention provides a stable pharmaceutical composition of an acid labile drug, comprising:
a) an inner core coated with the acid labile drug;
b) a first intermediate coating devoid of an alkaline stabilizing agent and the acid labile drug;
c) a second intermediate coating comprising an alkaline stabilizing agent; and
d) an outer enteric layer.

Preferably, the inner core is made of inert nonpareil sugar spheres. The acid labile drug, preferably, is a pharmaceutically active substituted benzimidazole compound. The pharmaceutically active substituted benzimidazole compound may include lansoprazole, omeprazole, pantoprazole, esomeprazole and rabeprazole. Preferably, the pharmaceutically active substituted benzimidazole compound is lansoprazole.

The first intermediate coating is devoid of an alkaline stabilizing agent and the acid labile drug; whereas the second intermediate coating comprises an alkaline stabilizing agent.

The present invention provides a stable pharmaceutical composition comprising an acid labile drug, preferably a pharmaceutically active substituted benzimidazole compound, that is resistant to dissolution in acidic dissolution media. However, the composition dissolves within 1 hour when the media is changed to an alkaline buffer.

The present invention provides a process of preparing a stable pharmaceutical composition of an acid labile drug such as a pharmaceutically active substituted benzimidazole compound, comprising the steps of:
a) coating an inner core with an aqueous suspension comprising the acid labile drug in the presence of an amine;
b) layering the inner core with a first intermediate coating;
c) layering the first intermediate coating with a second intermediate coating; and
d) layering the second intermediate coating with an outer enteric coating,
wherein the first intermediate coating is devoid of an alkaline stabilizing agent and the acid labile drug and the second intermediate coating comprises an alkaline stabilizing agent.

Preferably, the inner core is an inert sugar sphere. Preferably, the inner core has a diameter of about 850 to about 1,000 microns. Alternatively, larger inert sugar spheres of about 400 to about 500 microns are mixed with smaller inert sugar spheres of about 250 to about 350 microns in a weight ratio of about 2:1 to about 2.5:0.5 to form an inert sugar sphere mixture; and an inert sugar sphere from the inert sugar sphere mixture can be used as the inner core in another preferred embodiment of the present invention.

Preferably, the aqueous suspension in step a) further comprises hydroxypropyl methylcellulose and/or talc extra fine. Preferably, the amine in step a) exists as an aqueous amine solution in the aqueous suspension. Preferably, the acid labile drug is a pharmaceutically active substituted benzimidazole compound, more preferably, lansoprazole, and the amine is ammonia. More preferably, the amount of ammonia used in step a) constitutes about 0.005% to about 0.3% (w/w), preferably about 0.005% to about 0.03% (w/w), of the aqueous suspension used in step a), wherein the weight of the aqueous suspension includes the weight of the acid labile drug, water, ammonia and the optional hydroxypropyl methylcellulose and talc extra fine, but it excludes the weight of the inner core. Even more preferably, an aqueous ammonia solution of about 30% (v/v) is added to the aqueous suspension in step a) to provide the necessary amount of ammonia in step a). For instance, about 0.02% to about 0.1% (w/w) of the 30% (v/v) aqueous ammonia solution can be added to the aqueous suspension in step a).

Preferably, the first intermediate coating is layered by coating with a dispersion that comprises talc extra fine and hydroxypropyl methylcellulose.

Preferably, the second intermediate coating is layered by coating with a dispersion that comprises hydroxypropyl methylcellulose and magnesium carbonate.

Preferably, the outer enteric coating is layered by a dispersion that comprises talc extra fine, titanium dioxide, triethyl citrate and methacrylic acid copolymer. Different types of methacrylic acid copolymer can be used, and they include methacrylic acid copolymer type A (Eudragit® L-100), methacrylic acid copolymer type B (Eudragit® S-100), methacrylic acid copolymer type C (Eudragit® L 30D55, Eudragit® L-100-55) and a copolymer of methyacrylic acid methyl methacrylate and methyl methacrylate (Eudragit® FS).

The acid labile drug such as the pharmaceutically active substituted benzimidazole compound in the stable pharmaceutical composition of the present invention is in the form of particles which preferably have a 90^{th} volume percentile particle size of less than about 35 microns and a specific surface area of more than 0.5 m²/g.

### DETAILED DESCRIPTION OF THE INVENTION

The present invention provides a stable pharmaceutical composition comprising an acid labile drug such as a pharmaceutically active substituted benzimidazole compound where there is no physical contact between the acid labile drug and the second intermediate coating which contains an alkaline stabilizing agent. The pharmaceutical composition of the present invention has a good long-term stability.

In this patent application, the term “acid labile drug” refers to any drug, medicament or active pharmaceutical ingredient (API) that will degrade at a pH of 3. Examples of “acid labile drug” include pharmaceutically active substituted benzimidazole compounds, statins (e.g., pravastatin, fluvastatin and atorvastatin), antiobiotics (e.g., penicillin G, ampicillin, streptomycin, clarithromycin and azithromycin), dideoxyinosine (ddI or didanosine), dideoxyadenosine (ddA), dideoxycytosine (ddC), digoxin, pancreatin, bupropion and pharmaceutically acceptable salts thereof, such as bupropion HCl.

As used herein, the term “pharmaceutically active substituted benzimidazole compound” refers to any pharmaceutically active substituted 2-(2-pyridylmethyl)-sulfinyl-1H-benzimidazole compound (e.g., lansoprazole, omeprazole, hydroxyomeprazole, pantoprazole, rabeprazole, esomeprazole, perprazole, pariprazole and tenatoprazole) and pharmaceutically active substituted 2-(phenylmethyl)-sulfinyl-1H-benzimidazole compound (e.g., leminoprazole). The term “pharmaceutically active” means that the substituted benzimidazole compound has a pharmacological activity after being administered to the body of a subject, so “pharmaceutically active substituted benzimidazole compound” includes substituted benzimidazole compounds having a pharmacological activity directly or via certain activation mechanism, e.g. via hydrolysis yielding a pharmacologically active substance.

In accordance with the present invention, the stable pharmaceutical composition of the invention shows satisfactory stability under specified storage conditions. The stability of the composition is monitored, according to the pharmaceutical industry standard, under accelerated conditions of 40° C. and 75% relative humidity for three months. The term “stable” means that at least 90%, preferably at least 95%, more preferably at least 98% and most preferably at least 99%, by weight of the acid labile drug in the pharmaceutical composition remains after storage under accelerated conditions of 40° C. and 75% relative humidity for three months.

The stable pharmaceutical composition of the present invention can contain the acid labile drug or acid labile active pharmaceutical ingredient (API) (e.g., lansoprazole) in an amount of from about 2% to about 30% (w/w, based on the total weight of the inner core coated with the acid labile drug). Preferably, the weight of the acid labile drug is about 6% to about 16% of the total weight of the inner core coated with the acid labile drug. In an alternative embodiment, the weight of the acid labile drug is preferably about 18% to about 25% of the total weight of the inner core coated with the acid labile drug. The acid labile drug includes, but is not limited to, a pharmaceutically active substituted benzimidazole compound. Preferably, the pharmaceutically active substituted benzimidazole compound is lansoprazole.

Pharmaceutical Composition Comprising an Acid Labile Drug

(1) Inner Core Containing the Acid Labile Drug Such as a Pharmaceutically Active Substituted Benzimidazole Compound

The inner core is, preferably, made up of inert nonpareil (e.g., sugar spheres) spheres. The inert nonpareil spheres are exemplified by, but not limited to, sugar spheres, microcrystalline cellulose spheres, glass beads and coarse grade silicon dioxide cores. The inert sphere is about 45% to about 90% (w/w) of the inner core containing the acid labile drug. The inert sphere has a diameter of about 250 to about 1,200 microns; preferably the inert sphere has a diameter of about 850 to about 1,000 microns. Alternatively, in another preferred embodiment of the invention, inert sugar spheres of about 400 to about 500 microns are mixed with inert sugar spheres of about 250 to about 350 microns in a weight ratio of about 2:1 to about 2.5:0.5 to form an inert sugar sphere mixture, and an inert sugar sphere taken from the inert sugar sphere mixture is used as the inner core.

The inner core is coated with an aqueous suspension comprising the acid labile drug. The coating process is exemplified by a “Wurster”type column-equipped fluidized bed apparatus (i.e., Bottom spray technique). The aqueous suspension can comprise: 1) the acid labile drug in an amount of about 4% to about 30% (w/w) of the inner core coated with the acid labile drug; 2) a binder polymer in an amount of about 2% to about 16% (w/w) of the inner core coated with the acid labile drug; and 3) an anti-tackiness agent in an amount of about 2% to about 18% (w/w) of the inner core coated with the acid labile drug.

Preferably, a small amount of ammonia solution (in a concentration of about 30%, v/v) is added to the layer of the acid labile drug coating the inner core in order to impart an alkaline environment to the acid labile drug. Because ammonia is highly volatile during the coating processing, the final pharmaceutical composition does not contain any residue of ammonia.

Preferably, the acid labile drug is a pharmaceutically active substituted benzimidazole compound such as lansoprazole, omeprazole, pantoprazole, rabeprazole, pariprazole, perprazole, esomeprazole, hydroxyomeprazole, tenatoprazole or leminoprazole. More preferably, the acid labile drug is lansoprazole.

More preferably, the binder polymer is made up of one or more (i.e., mixtures) of hydroxypropyl methylcellulose, hydroxypropylcellulose, and polyvinyl alcohol. More preferably, the anti-tackiness agent is made up of one or more (i.e., mixtures) of talc, monoglycerides, diglycerides and magnesium stearate.

(2) First Intermediate Coating

The first intermediate coating is devoid of an alkaline stabilizing agent as well as an acid labile drug such as a pharmaceutically active substituted benzimidazole compound. Instead, the first intermediate layer comprises an inert polymer and an anti-tackiness agent. Preferably, the inert polymer is made up of one or more (i.e. mixtures) of binding agents. The binding agents are exemplified by hydroxypropyl methylcellulose, hydroxypropyl cellulose, and polyvinyl alcohol.

Additional examples of binding agents may include, but are not limited to, polyvinyl pyrrolidone, starch, methylcellulose, carboxymethyl cellulose, sucrose solution, and dextrose solution. The anti-tackiness agent is exemplified, but are not limited to, by talc, monoglycerides, diglycerides and magnesium stearate. Additional anti-tackiness agents may include, but are not limited to, silicon dioxide and metallic stearates.

The binding agent is sprayed from an aqueous or water-alcoholic suspension. Preferably, the binding agent is about 20% to about 85% (w/w) of the first intermediate layer. More preferably, the binding agent is about 30% to about 60% (w/w) of the first intermediate coating. Preferably, the anti-tackiness agent is about 15% to about 80% (w/w) of the first intermediate coating. More preferably, the anti-tackiness agent is about 40% to about 70% (w/w) of the first intermediate coating.

(3) Second Intermediate Coating

The second intermediate coating functions as a moisture barrier; in particular, as a buffering layer between the inner core containing the acid labile drug and the outer enteric layer. The second intermediate coating comprises an inert polymer and an alkaline stabilizing agent.

Preferably, the inert polymer is made up of one or more (i.e., mixtures) of a binding agent. The binding agent is exemplified by hydroxypropyl methylcellulose, hydroxypropyl cellulose and polyvinyl alcohol.

Additional examples of binding agents may include, but are not limited to, polyvinyl pyrrollidone, starch, methylcellulose, carboxymethyl cellulose, sucrose solution and dextrose solution.

Preferably, the alkaline stabilizing agent is made up of one or more (i.e., mixtures) of alkaline stabilizers exemplified, but not limited to, by magnesium carbonate, magnesium oxide, sodium hydroxide and organic bases such as TRIS (a.k.a THAM a.k.a tris(hydroxymethyl)aminomethane, (CH₂OH)₃CNH₂) and meglumine (1-deoxy-1-(methylamino)-D-glucitol). The second intermediate coating can be made by spraying an aqueous or water-alcohol suspension containing the necessary ingredients.

Additional examples of alkaline stabilizing agents may include, but are not limited to, magnesium hydroxide, magnesium metasilicate aluminate, magnesium silicate aluminate, magnesium silicate, magnesium aluminate, aluminum magnesium hydroxide, calcium carbonate, calcium hydroxide, potassium carbonate, sodium carbonate and sodium hydrogen carbonate.

Preferably, the inert polymer within the second intermediate coating is about 10% to about 70% (w/w) of the second intermediate coating. More preferably, the inert polymer within the second intermediate coating is about 35% to about 55% (w/w) of the second intermediate coating. Preferably, the alkaline stabilizer is about 30% to about 90% (w/w) of the second intermediate coating. More preferably, the alkaline stabilizer is about 45% to about 65% (w/w) of the second intermediate coating.

Preferably, the amount of the first intermediate coating and the amount of the second intermediate coating are, independently, about 2% to about 20% (w/w) of the inner core coated with the acid labile drug.

(4) Enteric Layer

The enteric layer usually comprises a polymer with enteric properties. The enteric polymer in the enteric layer is exemplified by methacrylic acid copolymer, hydroxypropyl methylcellulose phtalate and hydroxypropyl methylcellulose acetate succinate. Different types of methacrylic acid copolymer can be used, and they include methacrylic acid copolymer type A (Eudragit® L-100), methacrylic acid copolymer type B (Eudragit® S-100), methacrylic acid copolymer type C (Eudragit® L 30D55, Eudragit® L-100-55), a copolymer of methacrylic acid methyl methacrylate and methyl methacrylate (Eudragit® FS) and mixtures thereof, for instance, a mixture of Eudragit® L-100-55 and Eudragit® S-100 at a weight ratio of about 3:1 to about 2:1, or a mixture of Eudragit® L 30D55 and Eudragit® FS at a weight ratio of about 3:1 to about 5:1.

The enteric layer may further comprises other agents such as cellulose acetate phthalate, polyvinyl acetate phthalate, cellulose acetate trimellitate, shellac and/or zein.

Optionally, the enteric layer further comprises anti-tackiness agents such as talc or glyceryl monostearate; plasticizers such as triethylcitrate or polyethylene glycol; and pigments such as titanium dioxide or ferric oxides.

The enteric layer may further comprise one or more plasticizers including, but not limited to, acetyl triethyl citrate, acetyltributyl citrate, acetylated monoglycerides, glycerin, triacetin, propylene glycol, phthalate esters (e.g., diethyl phthalate, dibutyl phthalate), castor oil, sorbitol and dibutyl seccate.

Preferably, the enteric layer is about 5% to about 65% (w/w) of the stable pharmaceutical composition of the present invention. Preferably, the enteric polymer is about 50% to about 80% (w/w) of the enteric layer.

Preferably, the anti-tackiness agent is about 15 to about 60% (w/w) of the enteric layer. Preferably, the plasticizer is about 5 to about 20% (w/w) of the enteric layer. Preferably, the pigment is about 0.5 to about 10% (w/w) of the enteric layer.

The stable pharmaceutical composition of the present invention can be coated with one or more enteric layers, seal coatings, film coatings, barrier coatings, compression coatings, fast disintegrating coatings, or enzyme degradable coatings. Multiple coatings can be applied for desired performance.

Furthermore; the dosage form of the stable pharmaceutical composition of the invention can be designed for immediate release, pulsatile release, controlled release, extended release, delayed release, targeted release, synchronized release, or targeted delayed release. For release/absorption control, solid carriers can be made of various component types and levels or thickness of coats, with or without an active ingredient. Such diverse solid carriers can be blended in a dosage form to achieve a desired performance. The definitions of these terms are known to those skilled in the art. In addition, the dosage form release profile can be effected by a multiparticulate composition, a coated multiparticulate composition, an ion-exchange resin-based composition, an osmosis-based composition, or a biodegradable polymeric composition.

Without wishing to be bound by theory, it is believed that the release may be effected through favorable diffusion, dissolution, erosion, ion-exchange, osmosis or combinations thereof.

When the stable pharmaceutical composition of the invention is formulated as a capsule, the capsule can be a hard gelatin capsule, a starch capsule, or a cellulosic capsule. Although not limited to capsules, such dosage forms can further be coated with, for example, a seal coating, an enteric coating, an extended release coating, or a targeted delayed release coating. These various coatings are known in the art.

In one of the embodiments of the stable pharmaceutical composition of the present invention, the acid labile drug is particulate lansoprazole having a 90^{th} volume percentile particle size of less than about 35 microns and a specific surface area of more than 0.5 m²/g.

Preparation of the Pharmaceutical Composition: Coating Process

The coating process is exemplified by the following steps using a “Wurster” type column-equipped fluidized bed apparatus (Bottom spray technique). The sugar spheres of the inner core are preferably about 45 to about 90% (w/w) of the inner core coated with the acid labile drug. Preferably, the sugar spheres have a diameter of about 250 to about 1,200 microns. Alternatively, in another preferred embodiment of the invention, inert sugar spheres of about 400 to about 500 microns are mixed with inert sugar spheres of about 250 to about 350 microns in a weight ratio of about 2:1 to about 2.5:0.5 to form a mixture; and an inert sugar sphere from the mixture is used as the inner core. The inner core is coated with an aqueous suspension. The aqueous suspension comprises a) an acid labile drug such as a pharmaceutically active substituted benzimidazole compound in an amount of about 4 to about 30% (w/w) based on the total weight of the inner core coated with the acid labile drug; b) a binder polymer in an amount of about 2 to about 16% (w/w) based on the total weight of the inner core coated with the acid labile drug and c) an anti-tackiness agent in an amount of about 2 to about 18% (w/w) based on the total weight of the inner core coated with the acid labile compound.

It is known that coating processes of nonpareil cores with drug layers in fluidized bed apparatus can be very time consuming, especially when working at large scales. It was found that the addition of small quantities of a strong aqueous ammonia solution (wherein the concentration of the strong aqueous ammonia solution can be about 20% to about 40%, preferably ˜30%, v/v) to the drug layer imparts an alkaline environment to the active ingredient during processing. The added ammonia enhanced the stability of lansoprazole in the aqueous state and allowed spraying processes to continue upwards of 30 hours. Ammonia is known to be volatile and it evaporates during the coating process, so that it is not present in the final stable pharmaceutical composition. Therefore, when no alkalizing agents are to be present in the final sprayed layer, the aqueous suspension comprising the acid labile drug has to be temporarily stable until the layer is sprayed and the drug is deposited in dry state, which enhances its stability.

In order to obtain high drug potency by fluidized bed coating techniques, active pharmaceutical ingredient (API) particles having a specific surface area of more than 0.5 m²/g and a 90^{th} volume percentile particle size of less than about 35 microns are preferably used. “Specific surface area” represents the total particle surface, expressed in m² contained within 1 gram of particles of a given material and “90^{th} volume percentile” is defined as the diameter of particles below which 90% of the measured samples volume lies.

The present invention also provides a method of treating a disease selected from gastric or duodenal ulcer, severe erosive esophagitis, Zolinger-Ellison syndrome, gastroesophageal reflux and H. pylori infection, comprising administrating an effective amount of a stable pharmaceutical composition of the invention to a subject inflicted with the disease, preferably a subject in need of the treatment, wherein the acid labile drug in the stable pharmaceutical composition is selected from lansoprazole, omeprazole; pantoprazole, rabeprazole, hydroxyomeprazole, esomeprazole, pariprazole, perprazole, tenatoprazole, leminoprazole and pharmaceutically acceptable salts thereof.

The present invention is also directed toward a pharmaceutical composition of an acid labile drug, comprising an inner core coated with the acid labile drug, wherein the acid labile drug can degrade at pH 3, and wherein the acid labile drug is in a particulate form having a 90^{th} volume percentile particle size of less than about 35 microns and a specific surface area of more than 0.5 m²/g. Examples of the acid labile drug include the examples given for the stable pharmaceutical composition described above, with lansoprazole and its pharmaceutically acceptable salts being preferred. The present invention also provides a process of preparing the pharmaceutical composition of the acid labile drug, wherein the steps are as described for coating the inner core of the stable pharmaceutical composition with the acid labile drug.

The following non-limiting examples further illustrate the invention.

### EXAMPLE 1

A. Drug Layer (Inner Core Coated with Pharmaceutically Active Substituted Benzimidazole Compound)

Drug Layer Coating Suspension

3.3 kg of hydroxypropyl methylcellulose NF 6 cps was dispersed in 47.3 kg of purified water. 40 gms of strong ammonia solution (30%, v/v) were added. 3.3 kg talc extra fine was added and the solution was stirred. 6.6 kg lansoprazole was added and stirred until a homogeneous suspension was obtained. The homogeneous suspension was de-aerated overnight.

39.6 kg sugar spheres (850-1,000 micron) were introduced into a fluidized bed apparatus and the aforementioned suspension was sprayed onto the spheres. Then the spheres were dried, sifted through both a 14 mesh screen and a 30 mesh screen and were replaced into the fluidized bed apparatus for further coating.

B. Sub-Coat I (First Intermediate Coating)

Sub-Coat I Coating Suspension

0.8 kg of hydroxypropyl methylcellulose NF 6 cps was dispersed in 9.2 kg of purified water. 1.17 kg talc extra fine was homogenized in 2.25 kg purified water. The homogenized talc suspension was added to hydroxypropyl methylcellulose dispersion and stirred. The sub-coat suspension was sprayed onto 48 kg of drug layered pellets, i.e., the inner core coated with lansoprazole, hydroxypropyl methylcellulose and talc extra fine, from step A. The spheres were then dried, sifted through both a 14 mesh screen and a 30 mesh screen and replaced into the fluidized bed apparatus for further coating.

C. Sub-Coat II (Second Intermediate Coating)

Sub-Coat II Coating Suspension

1.5 kg of hydroxypropyl methylcellulose NF 6 cps was dispersed in 32.4 kg of purified water. 2.25 kg magnesium carbonate was added and stirred until a homogeneous suspension was obtained.

The sub-coat suspension was sprayed onto 47.5 kg of bi-layered pellets from step B. The spheres were then dried, sifted through both a 14 mesh screen and a 30 mesh screen and replaced into the fluidized bed apparatus for further coating.

D. Enteric Layer

Enteric Coating Dispersion

2.43 kg of talc extra fine, 0.27 kg of titanium dioxide and 0.54 kg of triethyl citrate were dispersed in 22.75 kg of purified water. 19.2 kg of methacrylic acid copolymer dispersion was added and stirred.

The enteric coating dispersion was sprayed onto 48.6 kg of spheres from step C. The spheres were then dried, sifted through both a 14 mesh screen and a 30 mesh screen and filled into hard gelatin capsules.

### EXAMPLE 2

Reference for Comparison (Alkaline Stabilizer Within Core)

A. Drug Layer (Inner Core Coated With Pharmaceutically Active Substituted Benzimidazole Compound)

Drug Layer Coating Suspension

3.9 kg of hydroxypropyl methylcellulose NF 6 cps was dispersed in 50.9 kg of purified water. 40 grams of a strong ammonia solution (30%, v/v) were added. 4.46 kg magnesium carbonate (MgCO₃) was added and stirred. 5.89 kg lansoprazole was added and stirred until a homogeneous suspension was obtained. The homogeneous suspension was de-aerated overnight.

35.1 kg of sugar spheres (850-1,000 micron) were introduced into a fluidized bed apparatus and the aforementioned suspension was sprayed onto the spheres. The spheres were then dried, sifted through both a 14 mesh screen and a 30 mesh screen and replaced into the fluidized bed apparatus for further coating.

B. Enteric Coating

Enteric Coating Dispersion

3.15 kg of talc extra fine, 0.35 kg of titanium dioxide and 0.7 kg of triethyl citrate were homogenized in 29.57 kg of purified water. 25.08 kg of methacrylic acid copolymer dispersion was added and stirred.

The enteric coating dispersion was sprayed onto 44.28 kg of drug coated spheres from the previous step. The spheres were then dried, sifted through both a 14 mesh screen and a 30 mesh screen and replaced into the fluidized bed apparatus for further coating.

### EXAMPLE 3

A. Drug Layer (Inner Core Coated with Pharmaceutically Active Substituted Benzimidazole Compound)

Drug Layer Coating Suspension

0.21 kg of hydroxypropyl methylcellulose NF 6 cps was dispersed in 3.0 kg of purified water. 4 grams of a strong ammonia solution (30%, v/v) were added. 0.21 kg talc extra fine was added and the solution was stirred. 0.55 kg lansoprazole was added and stirred until a homogeneous suspension was obtained. The homogeneous suspension was de-aerated.

0.65 kg sugar spheres (250-350 micron) and 0.33 kg sugar spheres (400-500 micron) were introduced into a fluidized bed apparatus and the aforementioned suspension was sprayed onto the spheres. Then the spheres were dried, sifted through both a 60 mesh screen and a 30 mesh screen and were replaced into the fluidized bed apparatus for further coating.

B. Sub-Coat I (First Intermediate Coating)

Sub-Coat I Coating Suspension

0.084 kg of hydroxypropyl methylcellulose NF 6 cps was dispersed in 0.87 kg of purified water. 0.13 kg talc extra fine was added and stirred. The sub-coat suspension was sprayed onto 0.68 kg of drug layered pellets, i.e., the inner core coated with lansoprazole and hydroxypropyl methylcellulose, and talc extra fine from stepA. The spheres were then dried, sifted through both a 60 mesh screen and a 25 mesh screen and replaced into the fluidized bed apparatus for further coating.

C. Sub-Coat II (Second Intermediate Coating)

Sub-Coat II Coating Suspension

0.21 kg of hydroxypropyl methylcellulose NF 6 cps was dispersed in 1.2 kg of purified water. 0.21 kg magnesium carbonate was added and stirred until a homogeneous suspension was obtained.

The sub-coat suspension was sprayed onto 1.87 kg of bi-layered pellets from step B. The spheres were then dried, sifted through both a 60 mesh screen and a 25 mesh screen and replaced into the fluidized bed apparatus for further coating.

D. Enteric Layer

Enteric Coating Dispersion

0.078 kg of talc extra fine, 0.016 kg of titanium dioxide and 0.02 kg of triethyl citrate were dispersed in 0.55 kg of acetone USP and 0.37 kg of isopropyl alcohol NF. 0.22 kg methacrylic acid copolymer (Eudragit® L-100-55) was dissolved in a mixture of 0.97 kg of acetone USP and 0.65 kg of isopropyl alcohol NF. The dispersion was added to the metacrylic acid copolymer solution and stirred.

The enteric coating dispersion was sprayed onto 0.63 kg of spheres from step C. The spheres were then dried, sifted through both a 60 mesh screen and a 20 mesh screen and filled into hard gelatin capsules or processed further for tabletting.

### EXAMPLE 4

A. Drug Layer (Inner Core Coated with Pharmaceutically Active Substituted Benzimidazole Compound)

Drug Layer Coating Suspension

0.21 kg of hydroxypropyl methylcellulose NF 6 cps was dispersed in 3.0 kg of purified water. 4 gms of strong ammonia solution (30% v/v) were added. 0.21 kg talc extra fine was added and the solution was stirred. 0.55 kg lansoprazole was added and stirred until a homogeneous suspension was obtained. The homogeneous suspension was de-aerated.

0.65 kg sugar spheres (250-350 micron) and 0.33 kg sugar spheres (400-500 micron) were introduced into a fluidized bed apparatus and the aforementioned suspension was sprayed onto the spheres. Then the spheres were dried, sifted through both a 60 mesh screen and a 30 mesh screen and were replaced into the fluidized bed apparatus for further coating.

B. Sub-Coat I (First Intermediate Coating)

Sub-Coat I Coating Suspension

0.084 kg of hydroxypropyl methylcellulose NF 6 cps was dispersed in 0.87 kg of purified water. 0.13 kg talc extra fine was added and stirred. The sub-coat suspension was sprayed onto 0.68 kg of drug layered pellets, i.e., the inner core coated with lansoprazole and hydroxypropyl methylcellulose, and talc extra fine from stepA. The spheres were then dried, sifted through both a 60 mesh screen and a 25 mesh screen and replaced into the fluidized bed apparatus for further coating.

C. Sub-Coat II (Second Intermediate Coating)

Sub-Coat II Coating Suspension

0.21 kg of hydroxypropyl methylcellulose NF 6 cps was dispersed in 1.2 kg of purified water. 0.21 kg magnesium carbonate was added and stirred until a homogeneous suspension was obtained.

The sub-coat suspension was sprayed onto 1.87 kg of bi-layered pellets from step B. The spheres were then dried, sifted through both a 60 mesh screen and a 25 mesh screen and replaced into the fluidized bed apparatus for further coating.

D. Enteric Layer

Enteric Coating Dispersion

0.09 kg of talc extra fine, 0.007 kg of titanium dioxide and 0. 0.03 kg of triethyl citrate were dispersed in 1.5 kg of purified water USP. 1.17 kg methacrylic acid copolymer dispersion (Eudragit® L-30 D-55) and 0.3 kg of a copolymer of methacrylic acid methyl methacrylate and methyl methacrylate (Eudragit® FS 30D) were mixed. The dispersion was added to the mixture of polymer dispersions and stirred.

The enteric coating dispersion was sprayed onto 0.63 kg of spheres from step C. The spheres were then dried, sifted through both a 60 mesh screen and a 20 mesh screen and filled into hard gelatin capsules or processed further for tabletting.

### EXAMPLE 5

A. Drug Layer (Inner Core Coated with Pharmaceutically Active Substituted Benzimidazole Compound)

Drug Layer Coating Suspension

0.21 kg of hydroxypropyl methylcellulose NF 6 cps was dispersed in 3.0 kg of purified water. 4 gms of a strong ammonia solution (30%, v/v) were added. 0.21 kg talc extra fine was added and the dispersion was stirred. 0.55 kg lansoprazole was added and stirred until a homogeneous suspension was obtained. The homogeneous suspension was de-aerated.

0.65 kg sugar spheres (250-350 micron) and 0.33 kg sugar spheres (400-500 micron) were introduced into a fluidized bed apparatus and the aforementioned suspension was sprayed onto the spheres. Then the spheres were dried, sifted through both a 60 mesh screen and a 30 mesh screen and were replaced into the fluidized bed apparatus for further coating.

B. Sub-Coat I (First Intermediate Coating)

Sub-Coat I Coating Suspension

0.084 kg of hydroxypropyl methylcellulose NF 6 cps was dispersed in 0.87 kg of purified water. 0.13 kg talc extra fine was added and stirred. The sub-coat suspension was sprayed onto 0.68 kg of drug layered pellets, i.e., the inner core coated with lansoprazole and hydroxypropyl methylcellulose, and talc extra fine from stepA. The spheres were then dried, sifted through both a 60 mesh screen and a 25 mesh screen and replaced into the fluidized bed apparatus for further coating.

C. Sub-Coat II (Second Intermediate Coating)

Sub-Coat II Coating Suspension

0.21 kg of hydroxypropyl methylcellulose NF 6 cps was dispersed in 1.2 kg of purified water. 0.21 kg magnesium carbonate was added and stirred until a homogeneous suspension was obtained.

D. Enteric Layer

Enteric Coating Dispersion

0.076 kg of talc extra fine, 0.007 kg of titanium dioxide and 0.022 kg of triethyl citrate were dispersed in 0.67 kg of alcohol 95% USP. 0.14 kg methacrylic acid copolymer (Eudragit® L-100-55) was dissolved in 1.44 Kg alcohol 95% USP. 0.058 kg methacrylic acid copolymer type B (Eudragit® S-100) was dissolved in 0.72 Kg alcohol 95% USP. The dispersion was added to the mixture of the methacrylic acid copolymer solution and stirred.

The enteric coating dispersion was sprayed onto 0.63 kg of spheres from step C. The spheres were then dried, sifted through both a 60 mesh screen and a 20 mesh screen and filled into hard gelatin capsules or processed further for tabletting.

Stability of the Final Pharmaceutical Formulation

The final pellet preparation was filled into gelatin capsules and was stored in high density polypropylene (HDPE) bottles of the following fill sizes: 30 caps (40 cc bottle), 100 caps (150 cc bottle) and 1,000 caps (1500 cc bottle).

These packaging types were submitted to accelerated storage conditions at 40° C. and 75% relative humidity. The results of the formulations are summarized in Table 1.

TABLE 1

| | Time zero | 3 months |
|---|---|---|
| Pellets (example 1) | 98.1* | Assay: 99.9* |
| 40 cc HDPE bottle | <0.1** | IDD: 0.1 |
| Pellets (example 1) | 98.1 | Assay: 98.3 |
| 150 cc HDPE bottle | <0.1 | IDD: 0.2 |
| Pellets (example 1) | 98.1 | Assay: 98.8 |
| 1,500 cc HDPE bottle | <0.1 | IDD: 0.6 |
| Pellets (Reference) | 99.7 | Assay: 96.5 |
| 40 cc HDPE bottle | 0.1 | IDD: 0.6 |
| Pellets (Reference) | 99.7 | Assay: 94.3 |
| 150 cc HDPE bottle | 0.1 | IDD: 0.8 |
| Pellets (Reference) | 99.7 | Assay: 95.5 |
| 1,500 cc HDPE bottle | 0.1 | IDD: 1.0 |

Assay refers to the assay measurement of lansoprazole by an in-house method.
HPLC Determination: Chromatographic System Column & Packaging: C18 (2);
Mobile Phase: water: acetonitrile: triethylamine 60:40:1 (v/v/v) adjusted to pH 7.0 ± 0.05, UV at 285 nm.
IDD represents “impurity degradation product” measured by the same HPLC in house method.
*Represents the amount of lansoprazole based on HPLC peak area.
**Represents the amount of impurity or degradation product in the lansoprazole based on HPLC peak area.

Table 1 demonstrates the superior stability of the stable pharmaceutical formulation of the present invention over the formulation containing an alkaline reacting compound in vicinity of the acid labile benzimidazole.

Results of long term stability studies of the stable pharmaceutical formulation of the present invention stored at 25° C. and 60% relative humidity, or 30° C. and 60% relative humidity are summarized in Tables 2 and 3, respectively.

TABLE 2

| | Time zero | 3 months | 6 months | 9 months | 12 months |
|---|---|---|---|---|---|
| Pellets (example 1) | Assay: 98.1 | Assay: 99.2 | Assay: 99.4 | Assay: 98.2 | Assay: 98.1 |
| 40 cc HDPE bottle | IDD: >0.1 | IDD: >0.1 | IDD: >0.1 | IDD: >0.1 | IDD: >0.1 |
| Pellets (example 1) | Assay: 98.1 | Assay: 98.0 | Assay: 100.1 | Assay: 99.7 | Assay: 99.9 |
| 150 cc HDPE bottle | IDD: >0.1 | IDD: >0.1 | IDD: >0.1 | IDD: >0.1 | IDD: >0.1 |
| Pellets (example 1) | Assay: 98.1 | Assay: 99.6 | Assay: 100.2 | Assay: 99.5 | Assay: 98.3 |
| 1500 cc HDPE bottle | IDD: >0.1 | IDD: >0.1 | IDD: >0.1 | IDD: 0.1 | IDD: 0.1 |

TABLE 3

| | Time zero | 3 months | 6 months | 9 months | 12 months |
|---|---|---|---|---|---|
| Pellets (example 1) | Assay: 98.1 | Assay: 98.9 | Assay: 99.5 | Assay: 100.3 | Assay: 98.8 |
| 40 cc HDPE bottle | IDD: >0.1 | IDD: >0.1 | IDD: >0.1 | IDD: >0.1 | IDD: >0.1 |
| Pellets (example 1) | Assay: 98.1 | Assay: 99.3 | Assay: 97.7 | Assay: 98.5 | Assay: |
| 150 cc HDPE bottle | IDD: >0.1 | IDD: >0.1 | IDD: >0.1 | IDD: 0.1 | 100.6 |
| | | | | | IDD: 0.1 |
| Pellets (example 1) | Assay: 98.1 | Assay: 98.1 | Assay: 98.5 | Assay: 98.8 | Assay: 97.6 |
| 1500 cc HDPE bottle | IDD: >0.1 | IDD: >0.1 | IDD: >0.1 | IDD: 0.2 | IDD: 0.2 |

Stability of the Drug Layer Coating Suspension

Preparation of the Drug Layer Coating Suspension:

Hydroxypropyl methylcellulose was dispersed in purified water and strong ammonia solution (30%, v/v) was added. Talc extra fine was then added and the suspension was stirred until homogeneity was obtained. Lansoprazole was added and stirred until homogeneous suspension was obtained. The homogeneous suspension was de-aerated overnight. Suspension samples were retrieved at the end of the preparation process (time zero) and at 10, 20, and 30 hours later.

Drug Potency: The drug potency of the suspension was measured by HPLC method, corresponding to the assay measurement method of the in-house lansoprazole monograph.

HPLC Determination: Chromatographic System Column & Packaging: C18 (2), Mobile Phase: Water:Acetonitrile:thiethylamine 60:40:1 (v/v/v) adjusted to pH 7.0±0.05, UV at 285 nm.

The results in Table 4 summarize the findings and demonstrate the stability of the drug layer coating suspension over a period of 30 hours.

TABLE 4

| | Drug potency in |
|---|---|
| Time | suspension* |
| Time zero | 99.8% |
| 10 hours | 99.9% |
| 20 hours | 99.6% |
| 30 hours | 99.2% |

*Based on the amount of lansoprazole added to the suspension.

Drug Potency, Particle Size and Specific Surface Area

The present invention provides an improved drug layering process. It was found that the particle size and specific surface area of the Active Pharmaceutical Ingredient (API) affected the drug layering process.

Malvernsizer S uses the volume of the particle to measure its size. For non-spherical and irregular particles, the diameter of an imaginary sphere that is equivalent in volume to the examined particle is calculated and the distribution derived. The results are presented as standard “percentile” readings: D(0.5), D(0.1) and D(0.9).

It is known to those skilled in the art that the drug layering process, performed with fluidized bed coating techniques, can yield low drug potency (assay). Low drug potency can result due to the possible combined effects of the phenomena of “spray drying” of the API solution/suspension before it reaches or adheres to the substrate, and/or abrasion of the drug (API) coated spheres du ring the layering process.

Using lansoprazole particles having a nominal diameter of below about 35 microns, it was found that such mean particle size improved the drug layering yield (Assay). Furthermore, it was found that characterization of the drug or API particles by their “size” is not enough to ensure that a high drug layering potency is obtained. API specific surface area is equally important. It was found that lansoprazole particles having a specific surface area of less than 0.5 m²/g and a 90^{th} volume percentile particle size of less than 35 microns did not yield the expected high potency.

All lansoprazole batches were measured by Malvern Laser Diffraction Mastersizer instrumentation model Mastersizer S. The Malvern Laser Diffraction Sizer uses the principle of light diffraction from particles in a liquid medium as the measurement means. The Mastersizer S can be used on particles ranging from 0.05 to 900 microns (laser λ=633 nm). The diffraction light pattern (He—Ne laser) is dependent on the particle size. The laser diffraction pattern is measured and correlated to the particle size distribution based on Fraunhofer or Mie theory. The use of Mie theory presupposes knowledge of the light refractive index of the particles and the dispersion media and the imaginary part of the refractive index of the particles.

The laser diffraction instrument Malvern Mastersizer 2000 has the following units: Flow-through cell for dispersion of particles in liquid media and small sample dispersion unit model DIF-2022 n in liquid media, Hydro μP, Dry dispenser for dispersion of particles in air, Scirroco 2000. The drug was dispersed in light liquid paraffin, and microscope evaluation was also performed. D(0.5) stands for the diameter of a particle larger than 50%, based on the total volume of all particles, of the particles in the particle sample. This value is also called Mass Median Diameter or Volume Median Diameter.

D(0.1) and D(0.9) are the diameters of particles below which 10% and 90% of the particle sample volume lie, respectively.

All lansoprazole batches were characterized by specific surface area measurement by Brunaver, Emmett and Teller (BET) method. The BET method is based on the adsorption of a condensable inert gas on the solid surface at reduced temperatures. Surface area obtained by the method provides information about the void spaces on the surface of the individual particles or aggregates. The BET surface equation is based on Langmur's kinetic theory of mono layer gas adsorption. BET expended the theory to multi molecular layer adsorption.

The instrument setup consists of a dewar containing a pure adsorbate (for example, nitrogen or krypton), carrier gas supply (helium), sample holder and detector. The sampler holder can allow the gas to flow, or a vacuum can be pulled on the sample.

Micromeritics Accelerated Surface Area and Porosity instrument ASAP 2000 with nitrogen as adsorbate was used. Specific surface area from abut 0.0005 m²/g (Kr) can be measured, no known upper limit. Pressure range: 0-950 mmHg. Vacuum system: two independent 2-stage mechanical pumps; one for analysis and one for degassing. Ultimate vacuum: 0.005 mm Hg. Nitrogen was used as analysis gas. Samples were kept in vacuum to room temperature overnight and than heated at 120° C. for 20 minutes. The samples were measured by single point BET method.

Drug Layering Procedure: a lansoprazole containing suspension is sprayed onto non-pareils (sugar sphere) with the aid of a fluidized bed technique, such as the Wurster-column equipped bottom spray procedure.

The drug potency (assay) was measured by the in-house method based on the lansoprazole USP monograph. HPLC Determination: Chromatograhic System Column & Packaging: C18 (2); Mobile Phase: Water:Acetonitrile:triethylamine 60;40:1 (v/v/v), adjusted to pH 7.0±0.05 with concentrated H₃PO₄, UV at 285 nm.

The effects of the particle size, and specific surface area upon obtained drug potency after the drug layering process are summarized in Table 5. They show that a particle size having D(0.9) less than 35 microns yields superior drug potencies of more than 95.0%. However, this rule does not include lots with low specific surface area (see Batch no. 6).

TABLE 5

| | | Specific Surface Area | Drug potency |
|---|---|---|---|
| Batch no. | Particle size | (m²/g) | (Assay %) |
| 1 | D(0.5) 16 micron | 0.3570 | 92.0% |
| | D(0.9) 49 micron | | |
| 2 | D(0.5) 16 micron | 0.3981 | 90.0% |
| | D(0.9) 47 micron | | |
| 3 | D(0.5) 15 micron | 0.5741 | 96.0% |
| | D(0.9) 34 micron | | |
| 4 | D(0.5) 9 micron | 0.5489 | 98.0% |
| | D(0.9) 24 micron | | |
| 5 | D(0.5) 3 micron | 1.0689 | 98.0% |
| | D(0.9) 10 micron | | |
| 6 | D(0.5) 14 micron | 0.2205 | 89.0% |
| | D(0.9) 29 micron | | |

The disclosures of the cited publications are incorporated herein in their entireties by reference. It is to be understood, however, that the scope of the present invention is not to be limited to the specific embodiments described above. The invention may be practiced other than as particularly described and still be within the scope of the accompanying claims.

1. A stable pharmaceutical composition of an acid labile drug, comprising:
a) an inner core coated with the acid labile drug;
b) a first intermediate coating devoid of an alkaline stabilizing agent and the acid labile drug;
c) a second intermediate coating comprising an alkaline stabilizing agent; and
d) an outer enteric layer,
wherein the acid labile drug can degrade at pH 3.

2. The stable pharmaceutical composition of claim 1, wherein the inner core is an inert sphere.

3. The stable pharmaceutical composition of claim 2, wherein the inert sphere is a nonpareil sugar sphere.

4. The stable pharmaceutical composition of claim 2, wherein the inert sphere has a weight of about 45% to about 90% of the total weight of the inner core coated with the acid labile drug.

5. The stable pharmaceutical composition of claim 2, wherein the inert sphere has a diameter of about 250 to about 1,200 microns.

6. The stable pharmaceutical composition of claim 5, wherein the inert sphere has a diameter of about 850 to about 1,000 microns.

7. The stable pharmaceutical composition of claim 1, wherein the inner core is an inert sugar sphere taken from a mixture of inert sugar spheres of about 400 to about 500 microns and inert sugar spheres of about 250 to about 350 microns mixed in a weight ratio of about 2:1 to about 2.5:0.5.

8. The stable pharmaceutical composition of claim 1, wherein the acid labile drug is a pharmaceutically active substituted benzimidazole compound.

9. The stable pharmaceutical composition of claim 8, wherein the pharmaceutically active substituted benzimidazole compound is selected from lansoprazole, omeprazole, pantoprazole, rabeprazole, hydroxyomeprazole, esomeprazole, pariprazole, perprazole, tenatoprazole, leminoprazole and pharmaceutically acceptable salts thereof.

10. The stable pharmaceutical composition of claim 9, wherein the pharmaceutically active substituted benzimidazole compound is lansoprazole or a pharmaceutically acceptable salt thereof.

11. The stable pharmaceutical composition of claim 1, wherein the inner core is coated with about 2% to about 30% (w/w, based on the total weight of the acid labile drug coated inner core) of the acid labile drug.

12. The stable pharmaceutical composition of claim 11, wherein the inner core is coated with about 6% to about 16% (w/w, based on the total weight of the acid labile drug coated inner core) of the acid labile drug.

13. The stable pharmaceutical composition of claim 11, wherein the inner core is coated with about 18% to about 25% (w/w, based on the total weight of the acid labile drug coated inner core) of the acid labile drug.

14. The stable pharmaceutical composition of claim 1, wherein the first intermediate coating comprises:
a) a binding agent comprising an inert polymer; and
b) an anti-tackiness agent.

15. The stable pharmaceutical composition of claim 14, wherein the binding agent is about 20% to about 85% (w/w) of the first intermediate coating.

16. The stable pharmaceutical composition of claim 14, wherein the anti-tackiness agent is about 15% to about 80% (w/w) of the first intermediate coating.

17. The stable pharmaceutical composition of claim 14, wherein the binding agent is at least one component selected from the group consisting of hydroxypropyl methylcellulose, hydroxypropylcellulose, polyvinyl alcohol, polyvinyl pyrrolidone, starch, carboxymethylcellulose, sucrose and dextrose.

18. The stable pharmaceutical composition of claim 14, wherein the anti-tackiness agent is at least one component selected from the group consisting of talc, monoglycerides, diglycerides and magnesium stearate.

19. The stable pharmaceutical composition of claim 1, wherein the second intermediate coating comprises:
a) an inert polymer; and
b) an alkaline stabilizing agent.

20. The stable pharmaceutical composition of claim 19, wherein the inert polymer is about 10% to about 70% (w/w) of the second intermediate coating.

21. The stable pharmaceutical composition of claim 20, wherein the inert polymer is about 35% to about 55% (w/w) of the second intermediate coating.

22. The stable pharmaceutical composition of claim 19, wherein the alkaline stabilizing agent is about 30% to about 90% (w/w) of the second intermediate coating.

23. The stable pharmaceutical composition of claim 22, wherein the alkaline stabilizing agent is about 45% to about 65% (w/w) of the second intermediate coating.

24. The stable pharmaceutical composition of claim 19, wherein the inert polymer is at least one component selected from the group consisting of hydroxypropyl methylcellulose, hydroxypropyl cellulose and polyvinyl alcohol.

25. The stable pharmaceutical composition of claim 19, wherein the alkaline stabilizing agent is at least one component selected from the group consisting of magnesium carbonate, magnesium oxide, sodium hydroxide, magnesium hydroxide, magnesium metasilicate aluminate, magnesium silicate aluminate, magnesium silicate, magnesium aluminate, aluminum magnesium hydroxide, calcium carbonate, calcium hydroxide, potassium carbonate, sodium carbonate, sodium hydrogen carbonate and an organic base.

26. The stable pharmaceutical composition of claim 25, wherein the organic base is tris(hydroxymethyl)aminomethane or 1-deoxy-1-(methylamino)-D-glucitol.

27. The stable pharmaceutical composition of claim 1, wherein the weight of the first intermediate coating is about 2% to about 20% of the total weight of the inner core coated with the acid labile drug.

28. The stable pharmaceutical composition of claim 1, wherein the weight of the second intermediate coating is about 2% to about 20% of the total weight of the inner core coated with the acid labile drug.

29. The stable pharmaceutical composition of claim 1, wherein the outer enteric layer comprises a polymer.

30. The stable pharmaceutical composition of claim 29, wherein the polymer is selected from methacrylic acid copolymer, hydroxypropyl methylcellulose phtalate, hydroxypropyl methylcellulose acetate succinate, methacrylic acid copolymer type A (Eudragit® L-100), methacrylic acid copolymer type B Eudragit® S-100), methacrylic acid copolymer type C (Eudragit®) L 30D55, Eudragit® L-100-55), a copolymer of methacrylic acid methyl methacrylate and methyl methacrylate (Eudragit® FS), and mixtures thereof.

31. The stable pharmaceutical composition of claim 30, wherein the polymer is a mixture of methacrylic acid copolymer type C (Eudragit® L-100-55) and methacrylic acid copolymer type B (Eudragit® S-100) at a weight ratio of about 3:1 to about 2:1, or a mixture of methacrylic acid copolymer type C (Eudragit® L 30D55) and a copolymer of methacrylic acid methyl methacrylate and methyl methacrylate (Eudragit® FS) at a weight ratio of about 3:1 to about 5:1.

32. The stable pharmaceutical composition of claim 29, wherein the polymer is about 50% to about 80% (w/w) of the outer enteric layer.

33. The stable pharmaceutical composition of claim 29, wherein the outer enteric layer further comprises a plasticizer.

34. The stable pharmaceutical composition of claim 33, wherein the plasticizer is triethylcitrate or polyethylene glycol.

35. The stable pharmaceutical composition of claim 33, wherein the plasticizer is about 5% to about 20% (w/w) of the polymer weight.

36. The stable pharmaceutical composition of claim 29, wherein the outer enteric layer further comprises an anti-tackiness agent.

37. The stable pharmaceutical composition of claim 36, wherein the anti-tackiness agent is about 15% to about 60% (w/w) of the outer enteric layer.

38. The stable pharmaceutical composition of claim 29, wherein the outer enteric layer further comprises a pigment.

39. The stable pharmaceutical composition of claim 38, wherein the pigment is titanium dioxide or ferric oxide.

40. The stable pharmaceutical composition of claim 39, wherein the pigment is about 0.5% to about 10% (w/w) of the polymer in the outer enteric layer.

41. The stable pharmaceutical composition of claim 29, wherein the weight of the outer enteric layer is about 5% to about 65% of the weight of the stable pharmaceutical composition.

42. The stable pharmaceutical composition of claim 1, wherein the stable pharmaceutical composition is stable under storage at 40° C. and 75% relative humidity for at least about 3 months.

43. The stable pharmaceutical composition of claim 1, wherein the stable pharmaceutical composition is in the form of a tablet, an ovule, a chewable tablet, a buccal tablet, a sub-lingual tablet, a granule, a pellet, a bead, or a pill.

44. The stable pharmaceutical composition of claim 43, wherein the stable pharmaceutical composition is in the form of an orally disintegrating tablet.

45. The stable pharmaceutical composition of claim 43, wherein the tablet comprises compressed beads.

46. The stable pharmaceutical composition of claim 1, wherein the stable pharmaceutical composition is formulated for immediate release, controlled release, extended release, delayed released, targeted release, or targeted delayed release.

47. The stable pharmaceutical composition of claim 1, wherein the acid labile drug is selected from pravastatin, fluvastatin, atorvastatin, penicillin G, ampicillin, streptomycin, clarithromycin, azithromycin, dideoxyinosine, dideoxyadenosine, dideoxycytosine, digoxin, pancreatin, bupropion, lansoprazole, omeprazole, pantoprazole, rabeprazole, hydroxyomeprazole, esomeprazole, pariprazole, perprazole, tenatoprazole, leminoprazole and pharmaceutically acceptable salts thereof.

48. The stable pharmaceutical composition of claim 1, wherein the acid labile drug is in a particulate form having a 90^{th} volume percentile particle size of less than about 35 microns and a specific surface area of more than 0.5 m²/g.

49. A pharmaceutical composition of an acid labile drug, comprising an inner core coated with the acid labile drug, wherein the acid labile drug can degrade at pH 3, and wherein the acid labile drug is in a particulate form having a 90^{th} volume percentile particle size of less than about 35 microns and a specific surface area of more than 0.5 m ²/g.

50. The pharmaceutical composition of claim 49, wherein the acid labile drug is selected from pravastatin, fluvastatin, atorvastatin, penicillin G, ampicillin, streptomycin, clarithromycin, azithromycin, dideoxyinosine, dideoxyadenosine, dideoxycytosine, digoxin, pancreatin, bupropion, lansoprazole, omeprazole, pantoprazole, rabeprazole, hydroxyomeprazole, esomeprazole, pariprazole, perprazole, tenatoprazole, leminoprazole and pharmaceutically acceptable salts thereof.

51. The pharmaceutical composition of claim 50, wherein the acid labile drug is lansoprazole or a pharmaceutically acceptable salt thereof.

52. The pharmaceutical composition of claim 49, wherein the inner core is an inert sphere.

53. The pharmaceutical composition of claim 52, wherein the inert sphere is a nonpareil sugar sphere.

54. The pharmaceutical composition of claim 52, wherein the inert sphere has a weight of about 45% to about 90% of the total weight of the inner core coated with the acid labile drug.

55. The pharmaceutical composition of claim 52, wherein the inert sphere has a diameter of about 250 to about 1,200 microns.

56. The pharmaceutical composition of claim 55, wherein the inert sphere has a diameter of about 850 to about 1,000 microns.

57. The pharmaceutical composition of claim 49, wherein the inner core is an inert sugar sphere taken from a mixture of inert sugar spheres of about 400 to about 500 microns and inert sugar spheres of about 250 to about 350 microns mixed in a weight ratio of about 2:1 to about 2.5:0.5.

58. A method of treating a disease selected from gastric or duodenal ulcer, severe erosive esophagitis, Zolinger-Ellison syndrome, gastroesophageal reflux or H. pylori infection, comprising administrating an effective amount of a stable pharmaceutical composition of claim 1 to a subject inflicted with the disease, wherein the acid labile drug in the stable pharmaceutical composition is selected from lansoprazole, omeprazole, pantoprazole, rabeprazole, hydroxyomeprazole, esomeprazole, pariprazole, perprazole, tenatoprazole, leminoprazole and pharmaceutically acceptable salts thereof.
